(19) United States
(12) Patent Application Publication
(10) Pub. No.: US 2020/0275840 A1
(43) Pub. Date: Sep. 3, 2020

(54) INFORMATION-PROCESSING APPARATUS, METHOD OF PROCESSING INFORMATION, AND MEDIUM

(71) Applicant: CANON KABUSHIKI KAISHA, Tokyo (JP)

(72) Inventors: Kenichi Nagae, Yokohama-shi (JP); Yoshitaka Baba, Tokyo (JP); Yukio Furukawa, Sagamihara-shi (JP)

(21) Appl. No.: 16/878,445

(22) Filed: May 19, 2020

Related U.S. Application Data
(63) Continuation of application No. PCT/JP2018/042691, filed on Nov. 19, 2018.

(30) Foreign Application Priority Data
Nov. 24, 2017 (JP) 2017-226009

(51) Int. Cl. A61B 5/00; A61B 5/1455; A61B 5/06

(52) U.S. Cl. CPC A61B 5/0095; A61B 5/7246; A61B 5/066; A61B 5/14551; A61B 5/0035

(57) ABSTRACT

An information-processing apparatus according to the present invention is an information-processing apparatus that processes information based on a photoacoustic wave that is generated from a subject by light emission to the subject. The information-processing apparatus includes image-data-acquiring means that acquires a plurality of image data for multiple times of light emission, based on a photoacoustic wave that is generated by the multiple times of light emission to the subject, template-data-acquiring means that acquires a plurality of template data for the multiple times of light emission, and correlation-information-acquiring means that acquires information about correlation between an image value column of the plurality of image data at a noteworthy position and a template data column of the plurality of template data at the noteworthy position.

### CROSS-REFERENCE TO RELATED APPLICATIONS

This application is a Continuation of International Patent Application No. PCT/JP2018/042691, filed Nov. 19, 2018, which claims the benefit of Japanese Patent Application No. 2017-226009, filed Nov. 24, 2017, both of which are hereby incorporated by reference herein in their entirety.

### BACKGROUND OF THE INVENTION

### Field of the Invention

The present invention relates to an information-processing apparatus that processes image data that is generated by a modality (image diagnosis device).

### Description of the Related Art

In a medical setting, diagnosis is made based on image data that is generated by a modality (image diagnosis device). An example of a known modality is an optoacoustic device that generates image data, based on a reception signal that is acquired by receiving an acoustic wave. The optoacoustic device emits pulsed light that is generated from a light source to a subject and receives an acoustic wave (typically, an ultrasonic wave, which is also referred to as a photoacoustic wave) that is generated from subject tissue that absorbs energy of the pulsed light propagating and diffusing in the subject. The optoacoustic device generates an image of subject information, based on the reception signal.

“Universal back-projection algorithm for photoacoustic computed tomography”, Minghua Xu and Lihong V. Wang, PHYSICAL REVIEW E 71, 016706 (2005)” discloses universal back-projection (UBP) that is a back-projection method as a method of generating an image of initial sound pressure distribution from a reception signal of a photoacoustic wave.

In the case where image data is generated by using a reception signal of an acoustic wave, an artifact appears in an image at a position that differs from a position at which the acoustic wave is generated. In some cases, it is consequently difficult to determine whether the image contains an image of a target (target for observation). Other than the optoacoustic device, another modality in which an artifact occurs has the same problem.

In view of this, it is an object of the present invention to provide an information-processing apparatus that acquires information that makes it easy to determine whether the possibility of the presence of a target (target for observation) at a noteworthy position in an image is high or low.

### SUMMARY OF THE INVENTION

An information-processing apparatus according to the present invention is an information-processing apparatus that processes information based on a photoacoustic wave that is generated from a subject by light emission to the subject. The information-processing apparatus includes image-data-acquiring means that acquires a plurality of image data for multiple times of light emission, based on a photoacoustic wave that is generated by the multiple times of light emission to the subject, and correlation-information-acquiring means that that acquires a plurality of template data for the multiple times of light emission, and that acquires information about correlation between an image value column of the plurality of image data at a noteworthy position and a template data column of the plurality of template data at the noteworthy position.

Further features of the present invention will become apparent from the following description of exemplary embodiments with reference to the attached drawings.

### BRIEF DESCRIPTION OF THE DRAWINGS

FIG. 1A is a diagram for a description of a time derivative process and a positive-negative inversion process of UBP.

FIG. 1B is a diagram for a description of the time derivative process and the positive-negative inversion process of the UBP.

FIG. 1C is a diagram for a description of the time derivative process and the positive-negative inversion process of the UBP.

FIG. 2A is a diagram for a description of a back-projection process of the UBP.

FIG. 2B is a diagram for a description of the back-projection process of the UBP.

FIG. 2C is a diagram for a description of the back-projection process of the UBP.

FIG. 2D is a diagram for a description of the back-projection process of the UBP.

FIG. 2E is a diagram for a description of the back-projection process of the UBP.

FIG. 3A illustrates a variation in an image value that is obtained by using the UBP.

FIG. 3B illustrates a variation in an image value that is obtained by using the UBP.

FIG. 4 illustrates images that are obtained by a process according to the present invention and by a process in a comparative example.

FIG. 5 illustrates a block diagram of an optoacoustic device according to an embodiment.

FIG. 6A schematically illustrates a probe according to the embodiment.

FIG. 6B schematically illustrates the probe according to the embodiment.

FIG. 7 illustrates a block diagram of a computer and peripheral components thereof according to the embodiment.

FIG. 8 illustrates the flow of a method of generating an image according to the embodiment.

FIG. 9 illustrates the flow of a process of generating image data according to the embodiment.

FIG. 10A illustrates partial volume data for multiple times of light emission according to the embodiment.

FIG. 10B illustrates partial volume data for multiple times of light emission according to the embodiment.

FIG. 10C illustrates partial volume data for multiple times of light emission according to the embodiment.

FIG. 11 illustrates template data according to the embodiment.

FIG. 12 illustrates a relationship among a light emission number, the partial volume data, an image value, and the template data according to the embodiment.

FIG. 13 is a graph illustrating changes in the image value of the partial volume data according to the embodiment.

FIG. 14A illustrates volume data and the template data according to the embodiment.

FIG. 14B illustrates volume data and the template data according to the embodiment.

FIG. 14C illustrates volume data and the template data according to the embodiment.

FIG. 14D illustrates volume data and the template data according to the embodiment.

### DESCRIPTION OF THE EMBODIMENTS

An embodiment of the present invention will hereinafter be described with reference to the drawings. However, the dimensions, materials, shapes, and relative positions of components described below are appropriately modified depending on the structure or various conditions of an apparatus for which the invention is used, and these do not limit the scope of the invention to the following description.

The present invention relates to processing of image data that represents two-dimensional or three-dimensional spatial distribution derived from a photoacoustic wave that is generated by light emission. Photoacoustic image data represents the spatial distribution of at least one piece of subject information such as sound pressure (initial sound pressure) when the photoacoustic wave is generated, light absorption energy density, a light absorption coefficient, and the concentration (such as oxygen saturation) of a substance of which a subject is composed.

An organism, which is a main subject of photoacoustic imaging, has light-scattering and light-absorbing properties. Accordingly, as light advances deep in the organism, light intensity exponentially attenuates. Consequently, there is a tendency that a photoacoustic wave having a high amplitude is generated near the surface of the subject and that a photoacoustic wave having a low amplitude is generated at a deep portion of the subject. In particular, a photoacoustic wave having a high amplitude is likely to be generated at a blood vessel near the surface of the subject.

In a reconstruction method called UBP (Universal Back-projection) disclosed in “Universal back-projection algorithm for photoacoustic computed tomography”, Minghua Xu and Lihong V. Wang, PHYSICAL REVIEW E 71, 016706 (2005)”, a reception signal is projected backward on an arc with a transducer centered. At this time, a reception signal of a photoacoustic wave having a high amplitude near the surface of the subject is projected backward on a deep portion of the subject. Consequently, an artifact occurs in the deep portion of the subject. For this reason, when organismic tissue in the deep portion of the subject is imaged, there is a possibility that the image quality (such as contrast) decreases due to the artifact caused by the photoacoustic wave that is generated from the surface of the subject.

According to the present invention, the quality of a photoacoustic image can be inhibited from decreasing due to the artifact. Processing according to the present invention will be described below.

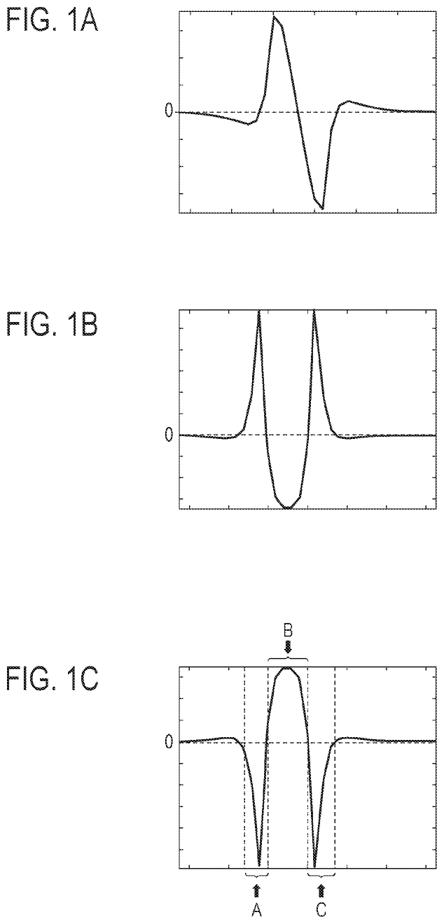

It is known that a reception signal of a photoacoustic wave typically has a waveform called N-Shape as illustrated in FIG. 1A. In the UBP, a time derivative process is performed on a N-Shape signal illustrated in FIG. 1A, and a time derivative signal illustrated in FIG. 1B is generated. Subsequently, a positive-negative inversion process is performed to inverse the positive and negative of a signal level of the time derivative signal, and a positive-negative inversion signal illustrated in FIG. 1C is generated. The signal (also referred to as a projection signal) that is generated by performing the time derivative process and the positive-negative inversion process on the N-Shape signal partly has a negative value as illustrated by arrows A and C in FIG. 1C and a positive value as illustrated in an arrow B in FIG. 1C.

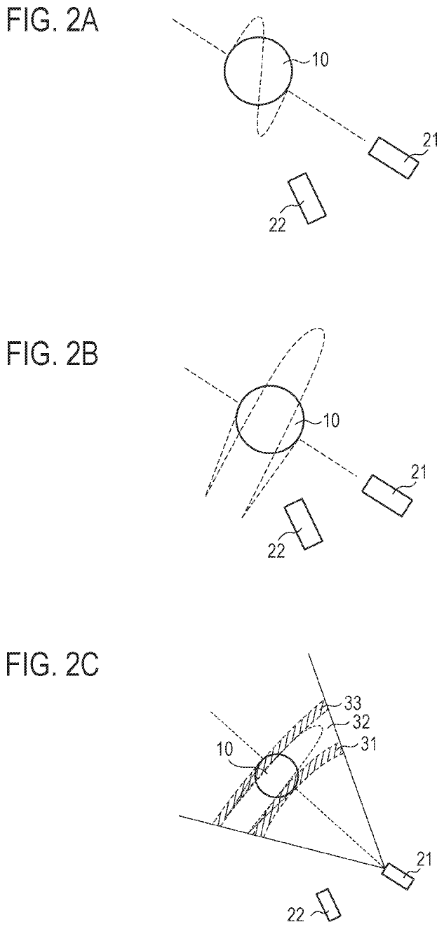

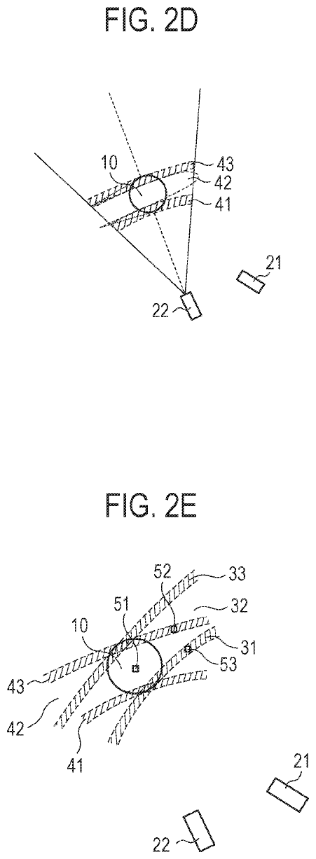

FIGS. 2A to 2E illustrate an example in which the UBP is used when a photoacoustic wave that is generated from a target 10 that is a minute, spherical light absorber in the subject is received by a transducer 21 and a transducer 22. When the target 10 is irradiated with light, the photoacoustic wave is generated. The photoacoustic wave is sampled as the N-Shape signal by the transducer 21 and the transducer 22. FIG. 2A illustrates a N-Shape reception signal that is sampled by the transducer 21 and that is superimposed on the target 10. For convenience, only the reception signal that is outputted from the transducer 21 is illustrated. However, a reception signal is outputted also from the transducer 22.

FIG. 2B illustrates the projection signal that is acquired by performing the time derivative process and the positive-negative inversion process on the N-Shape reception signal illustrated in FIG. 2A and that is superimposed on the target 10.

FIG. 2C illustrates a back-projection process of the UBP performed on the projection signal that is acquired by using the transducer 21. In the UBP, the projection signal is projected on an arc with the transducer 21 centered. In this case, the projection signal is projected backward within the range of the directional angle (for example, 60°) of the transducer 21. Consequently, an image as if the target 10 extends over regions 31, 32, and 33 is acquired. The regions 31 and 33 have negative values. The region 32 has a positive value. In FIG. 2C, the regions 31 and 33 having the negative values are filled with gray.

FIG. 2D illustrates the back-projection process of the UBP performed on the projection signal that is acquired by using the transducer 22. Consequently, an image as if the target 10 extends over regions 41, 42, and 43 is acquired. The regions 41 and 43 have negative values. The region 42 has a positive value. In FIG. 2D, the regions 41 and 43 having the negative values are filled with gray.

FIG. 2E illustrates the back-projection process of the UBP performed on the projection signals relative to the respective transducers 21 and 22. Photoacoustic image data is generated by combining the projection signals that are thus projected backward.

As illustrated in FIG. 2E, at a position 51 in the target 10, the region 32 having the positive value of the projection signal relative to the transducer 21 overlaps the region 42 having the positive value of the projection signal relative to the transducer 22. That is, in a region (also referred to as a target region) in which the target 10 is located, the regions having the positive values typically overlap superiorly. Accordingly, in the region in which the target 10 is located, there is a tendency that image data for every light emission typically has a positive value.

At a position 52 outside the target 10, the region 32 having the positive value of the projection signal relative to the transducer 21 overlaps the region 43 having the negative value of the projection signal relative to the transducer 22. At a position 53 outside the target 10, the region 31 having the negative value of the projection signal relative to the transducer 21 overlaps the region 41 having the positive value of the projection signal relative to the transducer 22. In the regions outside the target 10, there is thus a tendency that the region having the positive value and the regions having the negative values complexly overlap. That is, in the regions outside the target 10, there is a tendency that the image data for every light emission can have a positive value or a negative value. The reason for such a tendency is presumably that relative positions of the transducer 22 and the target 10 change for every light emission.

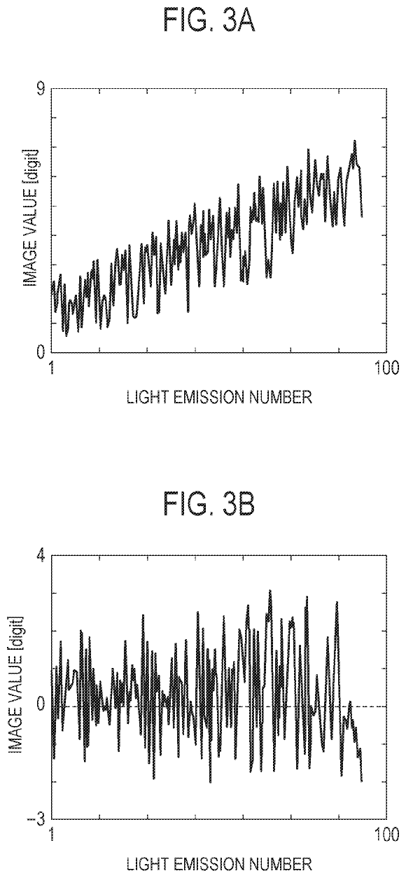

The following description pertains to variations in the value (image value) of the image data for every light emission when the combination of positions at which the photoacoustic wave is received is changed for every light emission. FIG. 3A illustrates the variation in the value (image value) of the image data of a region in the target 10 after reconstruction by the UBP disclosed in NPL 1. The horizontal axis represents a light emission number. The vertical axis represents the image value. FIG. 3B illustrates the variation in the value (image value) of the image data of a region outside the target 10 after reconstruction of the UBP disclosed in NPL 1. The horizontal axis represents the light emission number. The vertical axis represents the image value.

In FIG. 3A, it is seen that the image value of the region in the target 10 varies for every light emission but is always a positive value. In FIG. 3B, it is seen that the image value of the region outside the target 10 is a positive value or a negative value for every light emission.

Image data is generated by combining the image data for the whole of the light emission. In this case, the final image value of the region in the target 10 increases because positive values are combined. The final image value of the region outside the target 10 is smaller than that of the region in the target 10 because the positive value and the negative value of the image data are canceled out each other. Consequently, the presence of the target 10 can be visually recognized in an image based on the photoacoustic image data.

In some cases, however, the image value of the region outside the target 10 is not 0 even when there is no target therein, and the final image value is a positive value. In this case, an artifact that occurs at a position outside the target 10 decreases the visibility of the target.

Accordingly, there is a need to make it easy to determine whether an image of a region is an image of the target or an image of a non-target.

In view of this, the present inventor has conceived that variation characteristics of the image data for every light emission typically tend to differ between a region in the target and a region outside the target, and that this is used to solve the above problems. That is, the present inventor has conceived that the region in the target and the region outside the target are determined from the variation characteristics of the image value of the image data for every light emission.

The present inventor has also conceived that an image that represents the result of determination of the target region is displayed. Displaying such an image makes it easy to determine whether the target is located at a position in the image.

The present inventor has also conceived that the region in the target is determined by the above method, and that an image from which the image of the target is separated is generated from the image data. That is, the present inventor has conceived that, when the target is not located at a position, an image based on the image data at the position is displayed with luminance lower than luminance depending on the image value at the position. Such an image generation method enables a user to acquire an image in which the target is emphasized. Displaying such an image enables the user to readily determine whether the target is located at the position.

The present inventor has also conceived that an image based on characteristic information about the characteristics of a plurality of the image data for multiple times of light emission is displayed. Displaying such characteristic information enables the user to readily determine whether the target is located at the position.

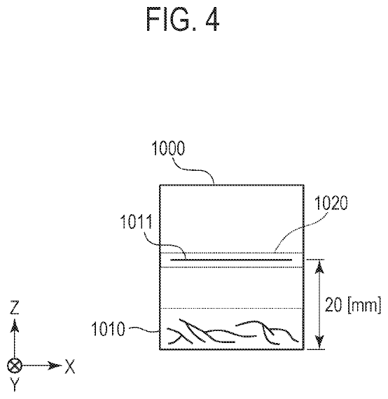

The processing according to the present invention will be described in detail later according to the embodiment below. In an example described according to the embodiment below, a reception signal of a photoacoustic wave is generated in a simulation with a subject model 1000 illustrated in FIG. 4, and image data is generated by using the reception signal. FIG. 4 illustrates the subject model 1000 used in the simulation. In the subject model 1000, a blood vessel 1010 is located near a surface, and a 0.2 [mm] blood vessel 1011 that extends in a Y-axis direction is located at a depth of 20 [mm] from the surface. The target in the subject model 1000 is each blood vessel. Reception signals, when photoacoustic waves that are generated from the blood vessels 1010 and 1011 in the subject model 1000 during multiple times of light emission are received by a receiver that is disposed below the subject model 1000 on the paper, are simulated. The reception signals are simulated by changing the position at which each photoacoustic wave is received for every light emission. A reconstruction process of the universal back-projection (UBP) described later is performed by using the reception signals that are acquired in the simulation, and the image data for the respective multiple times of light emission is generated.

In an example described according to the present embodiment, photoacoustic image data is generated by using an optoacoustic device. According to the present embodiment, the structure of the optoacoustic device and a method of processing information will now be described.

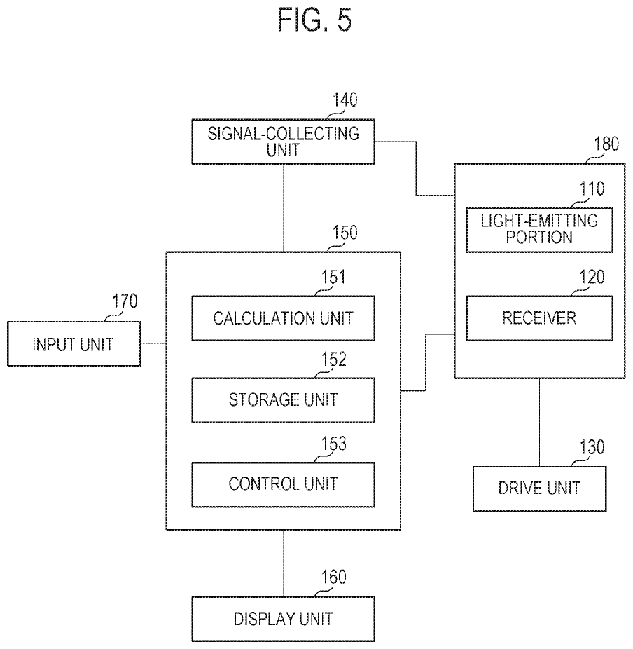

The structure of the optoacoustic device according to the present embodiment will be described with reference to FIG. 5. FIG. 5 schematically illustrates a block diagram of the entire optoacoustic device. The optoacoustic device according to the present embodiment includes a probe 180 that includes a light-emitting portion 110 and a receiver 120, a drive unit 130, a signal-collecting unit 140, a computer 150, a display unit 160, and an input unit 170.

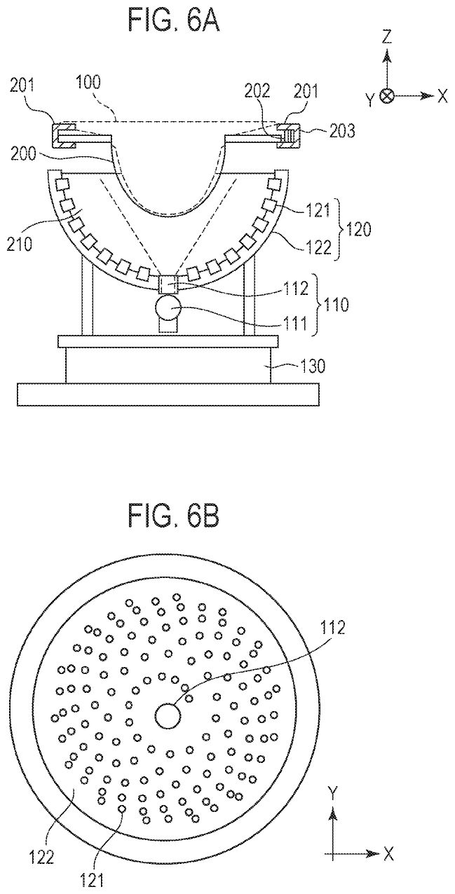

FIGS. 6A and 6B schematically illustrate the probe 180 according to the present embodiment. The target for measurement is a subject 100. The drive unit 130 drives the light-emitting portion 110 and the receiver 120 and mechanically scans. The light-emitting portion 110 irradiates the subject 100 with light, and an acoustic wave is generated in the subject 100. The acoustic wave that is generated by a photoacoustic effect caused by the light is also referred to as a photoacoustic wave. The receiver 120 receives the photoacoustic wave and outputs an electrical signal (photoacoustic signal) that is an analog signal.

The signal-collecting unit 140 converts the analog signal that is outputted from the receiver 120 into a digital signal and outputs the digital signal to the computer 150. The computer 150 stores the digital signal that is outputted from the signal-collecting unit 140 as signal data derived from the photoacoustic wave.

The computer 150 processes the stored digital signal to generate the photoacoustic image data that represents the two-dimensional or three-dimensional spatial distribution of information (subject information) about the subject 100. The computer 150 causes the display unit 160 to display an image based on the acquired image data. A doctor corresponding to the user can make diagnosis by checking the image that is displayed on the display unit 160. The displayed image is saved in a memory in the computer 150 or a memory of, for example, a data management system that is connected to a modality via a network in accordance with an instruction for saving from the user or the computer 150.

The computer 150 implements drive control of components that are included in the optoacoustic device. The display unit 160 may display a GUI in addition to the image that is generated by the computer 150. The input unit 170 enables the user to input information. The user can carry out an operation such as start or end of measurement or an instruction for saving of the generated image by using the input unit 170.

The structure of the optoacoustic device according to the present embodiment will now be described in detail.

(Light-Emitting Portion 110)

The light-emitting portion 110 includes a light source 111 that generates light and an optical system 112 that guides the light emitted from the light source 111 toward the subject 100. The light includes pulsed light such as a so-called square wave or a pyramidal wave.

The pulse width of the light that is generated from the light source 111 may be no less than 1 ns and no more than 100 ns. The wavelength of the light may range from about 400 nm to 1600 nm. In the case where a blood vessel is imaged with high resolution, the wavelength may be a wavelength (no less than 400 nm and no more than 700 nm) at which absorption at the blood vessel is good. In the case where a deep portion of an organism is imaged, the light may have a wavelength (no less than 700 nm and no more than 1100 nm) at which absorption at background tissue (water or fat) of the organism is typically poor.

An example of the light source 111 can be a laser or a light-emitting diode. In the case of measurement with light having multiple wavelengths, a light source that can change the wavelength may be used. In the case of irradiating the subject at multiple wavelengths, it is possible that multiple light sources that generate light at different wavelengths are prepared, and that the light sources alternately emit light. In the case of using the multiple light sources, the light sources are collectively referred to as the light source. Examples of the laser can include various lasers such as a solid-state laser, a gas laser, a dye laser, and a semiconductor laser. For example, the light source may be a pulse laser such as a Nd:YAG laser or an alexandrite laser. Alternatively, the light source may be an OPO (Optical Parametric Oscillators) laser or a Ti:sa laser that changes the light of the Nd:YAG laser into excitation light. Alternatively, the light source 111 may be a flash lamp or a light-emitting diode. Alternatively, the light source 111 may be a microwave source.

Optical elements such as a lens, a mirror, a prism, an optical fiber, a diffuser panel, and a shutter may be used as the optical system 112.

Regarding the permissible intensity of light with which organism tissue is irradiated, the maximum permissible exposure (MPE) is defined by the following safety standards (IEC60825-1: Safety of laser products, JIS C 6802: safety standards of laser products, FDA: 21CFR Part 1040.10, and ANSI Z136.1: Laser Safety Standards, etc.). The maximum permissible exposure prescribes the intensity of light that can be emitted per unit area. For this reason, a large amount of light can be guided to a subject E by irradiating the surface of the subject E with the light collectively from a large area, and the photoacoustic wave can be received at a high SN ratio. When the subject 100 is organism tissue of, for example, the breast, an emission portion of the optical system 112 may include, for example, a diffuser panel that causes light to diffuse in order to emit high-energy light having an increased beam diameter. In a photoacoustic microscope, a light-emitting portion of the optical system 112 may include, for example, a lens to emit a focused beam in order to increase resolution.

The light-emitting portion 110 may not include the optical system 112, and the subject 100 may be directly irradiated with the light from the light source 111.

(Receiver 120)

The receiver 120 includes transducers 121 each of which receives an acoustic wave and outputs an electrical signal, and a support 122 that supports the transducers 121. Each transducer 121 may be transmission means that transmits an acoustic wave. The transducer that serves as reception means and the transducer that serves as the transmission means may be a single (common) transducer or may be separated components.

The material of each transducer 121 can be a piezoelectric ceramic material represented by PZT (lead zirconate titanate) or a polymeric piezoelectric film material represented by PVDF (polyvinylidene fluoride). An element other than a piezoelectric element may be used. For example, a transducer using capacitive micro-machined ultrasonic transducers (CMUT) and a Fabry-Perot interferometer may be used. Provided that the electrical signal can be outputted by receiving the acoustic wave, any transducer may be used. The signal that is acquired by using each transducer is a time-resolved signal. That is, the amplitude of the signal that is acquired by the transducer represents a sound-pressure-based value (for example, a value in proportion to sound pressure) that is received by the transducer at a time.

The frequency component of the photoacoustic wave typically ranges from 100 KHz to 100 MHz. The transducers 121 can detect the frequency.

The support 122 may be composed of a metal material the mechanical strength of which is high. The surface of the support 122 facing the subject 100 may be processed to be a mirror surface or to be a surface that enables light to scatter such that the amount of emission light incident on the subject increases. According to the present embodiment, the support 122 has a hemispherical shape and can support the transducers 121 in the hemispherical shape. In this case, the directional axes of the transducers 121 that are disposed on the support 122 are concentrated on a position near the center of curvature of a hemisphere. When an image is generated by using the signals that are outputted from the transducers 121, the image quality near the center of curvature is high. The support 122 may have any structure, provided that the transducers 121 can be supported. On the support 122, the transducers may be arranged on a flat surface or a curved surface as called a 1 D array, a 1.5 D array, a 1.75 D array, or a 2 D array. For example, the transducers may be spirally arranged or may be arranged in Fibonacci sequence. The transducers 121 correspond to the reception means.

The support 122 may function as a container that stores an acoustic matching material 210. That is, the support 122 may be a container in which the acoustic matching material 210 is disposed between the transducers 121 and the subject 100.

The receiver 120 may include an amplifier that amplifies sequential analog signals that are outputted from the transducers 121. The receiver 120 may include an A/D converter that converts the sequential analog signals that are outputted from the transducers 121 into sequential digital signals. That is, the receiver 120 may include the signal-collecting unit 140 described later.

The transducers 121 is ideally arranged so as to surround the subject 100 from the whole circumference in order to detect the acoustic wave at various angles. In the case where the subject 100 is large, and the transducers cannot be arranged so as to surround the subject 100 from the whole circumference, the transducers may be disposed on the support 122 that has a hemispherical shape such that the subject 100 is surrounded from the whole circumference as much as possible.

The number of the transducers and the shape of the support may be optimized depending on the subject. According to the present invention, the receiver 120 can have any structure.

A space between the receiver 120 and the subject 100 is filled with a medium through which the photoacoustic wave can propagate. The medium can be a material through which the acoustic wave can propagate, and the material enables acoustic characteristics to be matched along the interface between the subject 100 and the transducers 121 and enables the transmittance of the photoacoustic wave to be increased. Examples of the medium can include water and ultrasonic gel.

FIG. 6A is a side view of the probe 180. FIG. 6B is a top view of the probe 180 (viewed from above the paper in FIG. 6A). The probe 180 according to the present embodiment illustrated in FIGS. 6A and 6B includes the receiver 120 in which the transducers 121 are three-dimensionally arranged on the support 122 that has an opening and a hemispherical shape. In the probe 180 illustrated in FIGS. 6A and 6B, the light-emitting portion of the optical system 112 is located at the bottom of the support 122.

According to the present embodiment, as illustrated in FIGS. 6A and 6B, the subject 100 is in contact with a holding portion 200, and the shape thereof is maintained. According to the present embodiment, in the case where the subject 100 is the breast, it is assumed that a bed that supports a subject person in a prone position has an opening in which the breast is inserted, and that the breast sagging in the vertical direction from the opening is measured.

The space between the receiver 120 and the holding portion 200 is filled with the medium (the acoustic matching material 210) through which the photoacoustic wave can propagate. The medium is a material through which the photoacoustic wave can propagate, and the material enables the acoustic characteristics to be matched along the interface between the subject 100 and the transducers 121 and enables the transmittance of the photoacoustic wave to be increased. Examples of the medium can include water, castor oil, and ultrasonic gel.

The holding portion 200 that serves as holding means is used to maintain the shape of the subject 100 during measurement. Since the subject 100 is held by the holding portion 200, the subject 100 is inhibited from moving, and the position of the subject 100 can be maintained in the holding portion 200. Examples of the material of the holding portion 200 can include resin materials such as polycarbonate, polyethylene, and polyethylene terephthalate.

The holding portion 200 is preferably composed of a material that has hardness enabling the subject 100 to be held. The holding portion 200 may be composed of a material through which the light used for measurement passes. The holding portion 200 may be composed of a material that has the same level of impedance as that of the subject 100. In the case where the subject 100 has a curved surface as in the breast, the holding portion 200 may be hollowed. In this case, the subject 100 can be inserted into a hollow of the holding portion 200.

The holding portion 200 is mounted on an attachment 201. The attachment 201 may enable various kinds of the holding portions 200 to be replaced with each other depending on the size of the subject. For example, the attachment 201 may enable the holding portion 200 with a holding portion having a different radius of curvature or a different center of curvature.

In the holding portion 200, a tag 202 in which information about the holding portion 200 is registered may be installed. For example, information about the identification ID, acoustic velocity, center of curvature, and radius of curvature of the holding portion 200 may be registered in the tag 202. The information that is registered in the tag 202 is read by a reading unit 203 and sent to the computer 150. In order to readily read the tag 202 when the holding portion 200 is mounted on the attachment 201, the reading unit 203 may be included in the attachment 201. For example, the tag 202 is a barcode, and the reading unit 203 is a barcode reader.

(Drive Unit 130)

The drive unit 130 changes the relative positions of the subject 100 and the receiver 120. According to the present embodiment, the drive unit 130 is a device that moves the support 122 in an XY direction and is an electric XY stage that includes a stepper motor. The drive unit 130 includes a motor such as a stepper motor that applies a driving force, a driving mechanism that transmits the driving force, and a position sensor that detects information about the position of the receiver 120. Examples of the driving mechanism can include a lead screw mechanism, a linkage mechanism, a gear mechanism, and a hydraulic mechanism. Examples of the position sensor can include a potentiometer using an encoder, a variable resistor, a linear scale, a magnetic sensor, an infrared sensor, or an ultrasonic sensor.

The drive unit 130 may not change the relative positions of the subject 100 and the receiver 120 in the XY direction (two-dimensionally) but may change the relative positions one-dimensionally or three-dimensionally. A movement path may be scanned in a plane spirally or in a line and space manner or may be three-dimensionally inclined along a body surface. The probe 180 may be moved such that a distance from the surface of the subject 100 is kept constant. At this time, the drive unit 130 may measure the amount of movement of the probe by monitoring the rotational speed of the motor.

The receiver 120 may be secured, and the subject 100 may be moved, provided that the drive unit 130 can change the relative positions of the subject 100 and the receiver 120. In the case where the subject 100 is moved, it is thought that the holding portion that holds the subject 100 is moved to move the subject 100. Both of the subject 100 and the receiver 120 may be moved.

The drive unit 130 may change the relative positions continuously or in a step and repeat manner. The drive unit 130 may be an electric stage in which the path of movement is programmed or may be a manually operated stage. That is, the optoacoustic device may be a hand-held device that does not include the drive unit 130 and that is operated in a manner in which the user grips the probe 180.

According to the present embodiment, the drive unit 130 simultaneously drives the light-emitting portion 110 and the receiver 120 for scanning but may drive only the light-emitting portion 110 or may drive only the receiver 120.

(Signal-Collecting Unit 140)

The signal-collecting unit 140 includes the amplifier that amplifies electrical signals that are the analog signals that are outputted from the transducers 121 and the A/D converter that converts the analog signals that are outputted from the amplifier into the digital signals. The signal-collecting unit 140 may include a FPGA (Field Programmable Gate Array) chip. The digital signals that are outputted from the signal-collecting unit 140 are stored in a storage unit 152 of the computer 150. The signal-collecting unit 140 is also referred to as a data acquisition system (DAS). In the present specification, the idea of an electrical signal includes an analog signal and a digital signal. An optical detection sensor such as a photo diode may detect light emission from the light-emitting portion 110, and the signal-collecting unit 140 may start the above processing synchronously such that the result of detection triggers. The signal-collecting unit 140 may start the above processing synchronously such that an instruction given by using, for example, a freeze button triggers.

(Computer 150)

The computer 150 that serves as an information-processing apparatus includes a calculation unit 151, the storage unit 152, and a control unit 153. The function of each component will be described together with a description of a process flow.

A unit that serves as the calculation unit 151 and that has a calculation function can include a processor such as a CPU or a GPU (Graphics Processing Unit) and a calculation circuit such as a FPGA (Field Programmable Gate Array) chip. The unit may include processors and calculation circuits instead of the single processor and the single calculation circuit. The calculation unit 151 may receive various parameters such as the acoustic velocity in the subject and the structure of the holding portion from the input unit 170 to process a reception signal.

The storage unit 152 may include a non-temporary storage medium such as a ROM (Read only memory), a magnetic disk, or a flash memory. The storage unit 152 may be a volatile medium such as a RAM (Random Access Memory). The storage unit 152 may be a storage server such as a PACS (Picture Archiving and Communication System) via a network. A storage medium that stores a program is the non-temporary storage medium. The storage unit 152 may include storage media instead of the single storage medium.

The storage unit 152 can save image data that represents a photoacoustic image that is generated by the calculation unit 151 in a manner described later.

The control unit 153 includes a calculation element such as a CPU. The control unit 153 controls the operation of each component of the optoacoustic device. The control unit 153 may receive instruction signals given by various kinds of operation such as start of measurement from the input unit 170 to control each component of the optoacoustic device. The control unit 153 reads out a program code that is stored in the storage unit 152 to control the operation of each component of the optoacoustic device. For example, the control unit 153 may control the timing of the light emission of the light source 111 by using a control line. In the case where the optical system 112 includes a shutter, the control unit 153 may control open and close of the shutter by using a control line.

The computer 150 may be a workstation that is exclusively designed. The components of the computer 150 may include different pieces of hardware. At least a part of the structure of the computer 150 may be a single piece of hardware.

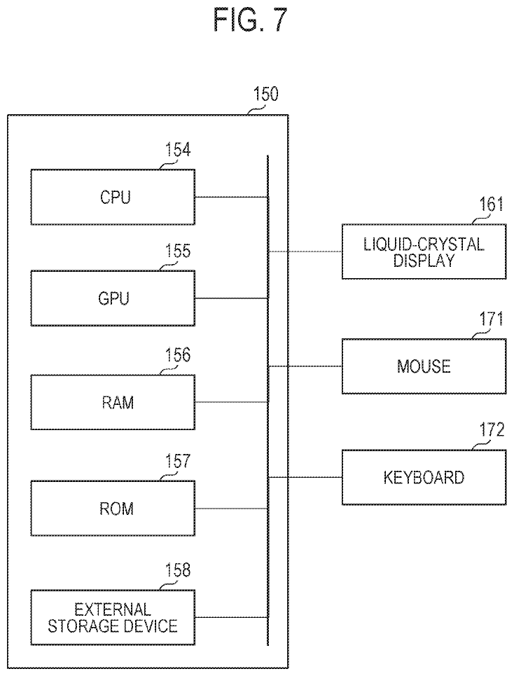

FIG. 7 illustrates a specific example of the computer 150 according to the present embodiment. According to the present embodiment, the computer 150 includes a CPU 154, a GPU 155, a RAM 156, a ROM 157, and an external storage device 158. A liquid-crystal display 161 that serves as the display unit 160 and a mouse 171 and a keyboard 172 that serve as the input unit 170 are connected to the computer 150.

The computer 150 and the transducers 121 may be contained in a common housing. Some signal processes may be performed by the computer that is contained in the housing, and the other signal processes may be performed by a computer that is disposed outside the housing. In this case, the computers that are disposed inside and outside the housing can be collectively referred to as the computer according to the present embodiment. That is, hardware of the computer may not be contained in the housing.

(Display Unit 160)

The display unit 160 is a display such as a liquid-crystal display, an organic EL (Electro Luminescence) FED, an eyewear display, or a head-mounted display and is a device that displays, for example, an image based on volume data that is acquired by the computer 150 or the numeral of a specific position. The display unit 160 may display a GUI for operating the image based on the volume data or the device. The subject information can be displayed after the display unit 160 or the computer 150 performs an imaging process (such as adjustment of a luminance value). The display unit 160 may be provided separately from the optoacoustic device. The computer 150 can transmit the photoacoustic image data to the display unit 160 by using a wired cable or wireless.

(Input Unit 170)

The input unit 170 can be an operation console that includes a mouse and a keyboard that are operable by the user. The display unit 160 may include a touch screen, and the display unit 160 may be used as the input unit 170.

Through the input unit 170, information about a position or a depth of a portion to be observed may be inputted. An input method may be to input a numeral or to operate a slide bar. The image that is displayed on the display unit 160 may be updated depending on the inputted information. Consequently, the user can set an appropriate parameter while seeing an image that is generated by using a parameter that is determined by operation of the user.

The user may operate the input unit 170 that is disposed far from the optoacoustic device, and information that is inputted by using the input unit 170 may be transmitted to the optoacoustic device via a network.

The components of the optoacoustic device may be separated components or may be an integrated device. At least some of the components of the optoacoustic device may be an integrated device.

Information is received and transmitted between the components of the optoacoustic device by using a wired cable or wirelessly.

(Subject 100)

The subject 100, which is not included in the optoacoustic device, will now be described. The optoacoustic device according to the present embodiment can be used to diagnose a malignant tumor or vascular disease of a human being or an animal or to observe chemotherapy. Accordingly, the subject 100 that is assumed is an organism, specifically, the breasts, the internal organ, the vascular plexus, the head, the neck, the abdomen, the fingers, or the toes of a human body or an animal. For example, when the human body is a measurement target, the target for a light absorber may be oxyhemoglobin, deoxyhemoglobin, a blood vessel that includes these in a large amount, or a new blood vessel near a tumor. The target for the light absorber may be a plaque of the cervical artery wall. The target for the light absorber may be melanin, collagen, or a lipid that is contained in, for example, the skin. The light absorber may be pigment such as methylene blue (MB) or indocyanine green (ICG), a gold granule, or an externally introduced substance that is an accumulation thereof or that is chemically modified. The subject 100 may be a phantom that imitates an organism.

In the present specification, the above light absorber to be imaged is referred to as the target. A light absorber that is not to be imaged, that is, a light absorber that is not the target for observation is not the target. For example, in the case where the subject is the breasts, and the light absorber as the target is a blood vessel, it can be thought that tissue such as fat or mammary glands in the breasts is not the target. In the case where the target is a blood vessel, it is thought that light having a wavelength suitable for light absorption of the blood vessel is used.

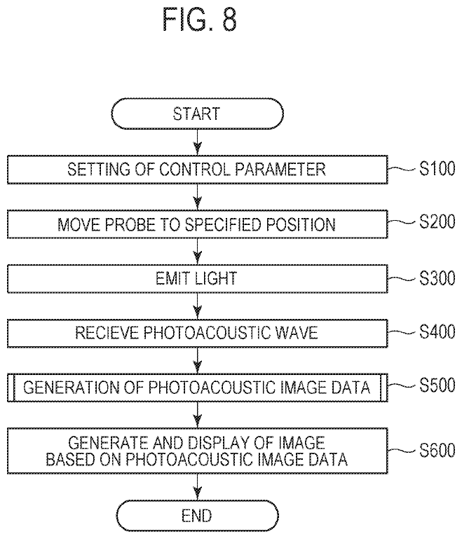

A method of displaying an image including information processing according to the present embodiment will now be described with reference to FIG. 8. Processes are performed by controlling the operation of each component of the optoacoustic device by the computer 150.

(S100: Process of Setting Control Parameter)

The user uses the input unit 170 to specify a control parameter, such as the position of the probe 180, and emission conditions (such as repetition frequency and the wavelength) of the light-emitting portion 110 that are needed to acquire the subject information. The computer 150 sets the control parameter that is determined based on the specification of the user.

(S200: Process of Moving Probe to Specified Position)

The control unit 153 causes the drive unit 130 to move the probe 180 to a specified position, based on the control parameter that is specified at S100. In the case where imaging at multiple positions is specified at S100, the drive unit 130 first moves the probe 180 to a first specified position. The drive unit 130 may move the probe 180 to a position that is programmed in advance in response to an instruction for start of measurement. In the case of the hand-held device, the user may move the probe 180 to a desired position while gripping.

(S300: Process of Emitting Light)

The light-emitting portion 110 irradiates the subject 100 with light, based on the control parameter that is specified at S100.

The light that is generated from the light source 111 passes through the optical system 112 into pulsed light with which the subject 100 is irradiated. The pulsed light is absorbed in the subject 100, and the photoacoustic wave is generated by the photoacoustic effect. The light-emitting portion 110 transmits a synchronous signal to the signal-collecting unit 140 at the same time the pulsed light propagates.

(S400: Process of Receiving Photoacoustic Wave)

The signal-collecting unit 140 starts collecting signals when receiving the synchronous signal that is transmitted from the light-emitting portion 110. That is, the signal-collecting unit 140 generates amplified digital electrical signals by amplification and A/D conversion of analog electrical signals that are derived from the acoustic wave and that are outputted from the receiver 120 and outputs the signals to the computer 150. The computer 150 saves the signals that are transmitted from the signal-collecting unit 140 in the storage unit 152. In the case where imaging at multiple scan positions is specified at S100, at the specified scan positions, the processes at S200 to S400 are repeatedly performed, the pulsed light is repeatedly emitted, and the digital signals that are derived from the acoustic wave are repeatedly generated. The computer 150 may acquire and store the information about the position of the receiver 120 at light emission such that the light emission triggers, based on the output from a position sensor of the drive unit 130.

(S500: Process of Generating Photoacoustic Image Data)

The calculation unit 151 of the computer 150 that serves as image-data-acquiring means generates the photoacoustic image data, based on signal data that is stored in the storage unit 152 and saves the photoacoustic image data in the storage unit 152.

Examples of a reconstruction algorithm that converts the signal data into volume data as spatial distribution can include analytic reconstruction methods such as a back-projection method in time domain and a back-projection method in Fourier domain, and a model-based method (iterative calculation method). Examples of the back-projection method in time domain include the universal back-projection (UBP), filtered back-projection (FBP), and delay-and-sum.

The calculation unit 151 may acquire information about absorption coefficient distribution in a manner in which light fluence distribution of the light with which the subject 100 is irradiated in the subject 100 is calculated, and initial sound pressure distribution is divided by the light fluence distribution. In this case, the information about the absorption coefficient distribution may be acquired as the photoacoustic image data. The computer 150 can calculate the spatial distribution of light fluence in the subject 100 by using a method of numerically solving a transport equation or a diffusion equation that represents the behavior of light energy in a medium in which the light is absorbed or scatters. Examples of the method of numerically solving the equation can include a finite element method, a difference method, and a Monte Carlo method. For example, the computer 150 may calculate the spatial distribution of the light fluence in the subject 100 by solving a light diffusion equation defined as Expression (1).

$$\begin{matrix}[ Expression 1 ] & \\ \frac{1}{c} \frac{\partial }{\partial t} φ ( r , t ) = - {μ}_{a} φ ( r , t ) + ∇ \cdot ( D ∇ φ ( r , t ) ) + S ( r , t ) & Expression ( 1 )\end{matrix}$$

Here, D is a diffusion coefficient, μa is an absorption coefficient, S is the intensity of incident emission light, ϕ is reaching light fluence, r is a position, and t is time.

The processes at S300 and S400 may be performed by using light having multiple wavelengths, and the calculation unit 151 may acquire the information about the absorption coefficient distributions relative to the light having the respective wavelengths. The calculation unit 151 may acquire, as the photoacoustic image data, spectral information about the spatial distribution of the concentration of the substance of which the subject 100 is composed, based on the information about the absorption coefficient distributions relative to the light having the respective wavelengths. That is, the calculation unit 151 may acquire the spectral information by using signal data relative to the light having the multiple wavelengths.

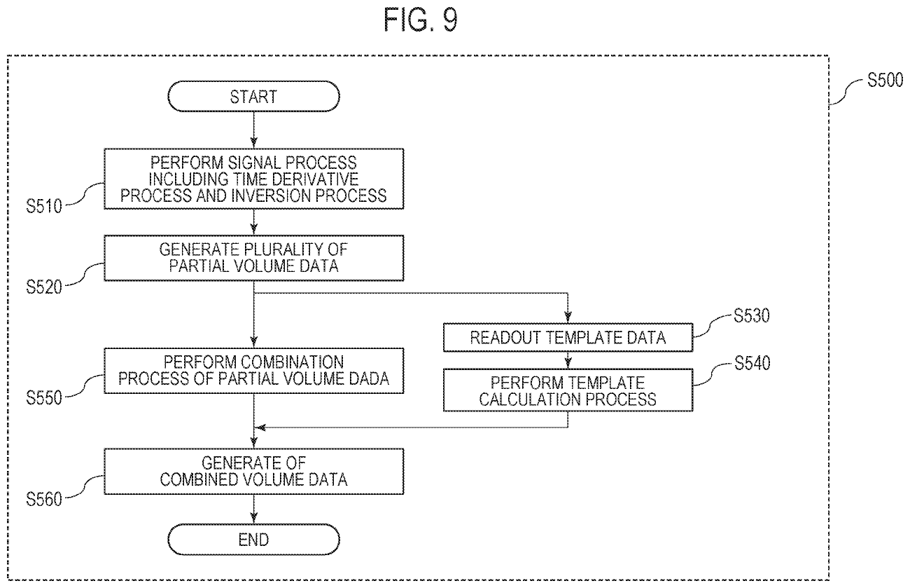

The detail of this process will be described later with reference to a flowchart in FIG. 9.

(S600: Process of Generating and Displaying Image Based on Photoacoustic Image Data)

The computer 150 that serves as display-controlling means generates an image, based on the photoacoustic image data that is acquired at S500 and causes the display unit 160 to display the image. The image value of the image data may be the luminance value of the image to be displayed. A predetermined process may be performed on the image value of the image data to determine the luminance of the image to be displayed. For example, the image to be displayed may be generated such that the luminance is set to the image value in the case where the image value is a positive value, and the luminance is set to 0 in the case where the image value is a negative value.

A method of generating the image at S500 will now be described with reference to a flowchart illustrated in FIG. 9. At S500, the computer 150 generates the photoacoustic image data, based on the signal data that contains the digital signals derived from the photoacoustic wave, acquired for every light emission.

(S510: Process of Performing Time Derivative Signal and Inversion Process on Reception Signal)

The computer 150 that serves as signal-processing means performs a signal process including a time derivative process and an inversion process to invert the positive and negative of the signal level on the reception signals that are stored in the storage unit 152. The reception signals on which the signal process is performed are referred to as projection signals. In this process, the signal process is performed on each reception signal that is stored in the storage unit 152. Consequently, projection signals are generated for the respective multiple times of light emission and the respective transducers 121.

For example, the computer 150 performs the time derivative process and the inversion process (the time derivative signal becomes negative) on a reception signal p (r, t) to generate a projection signal b (r, t) as defined in Expression (2) and stores the projection signal b (r, t) in the storage unit 152.

$$\begin{matrix}[ Expression 2 ] & \\ b ( r , t ) = 2 p ( r , t ) - 2 t \frac{\partial p ( r , t )}{\partial t} & Expression ( 2 )\end{matrix}$$

Here, r is the position of reception, t is elapsed time after the light emission, p (r, t) is the reception signal that represents the sound pressure of the acoustic wave that is received at the position r of reception and at the elapsed time t, and b (r, t) is the projection signal. Another signal process may be performed in addition to the time derivative process and the inversion process. For example, the other signal process includes frequency filtering (such as low pass, high pass, and band pass), deconvolution, envelope detection, or wavelet filtering, or all of these.

According to the present embodiment, the inversion process may not be performed. Also, in this case, the effects of the present embodiment are achieved.

(S520: Process of Generating Image Data Based on Reception Signal after Signal Process)

The computer 150 that serves as image-data-generating means generates a plurality of the photoacoustic image data, based on the reception signals (projection signals) that are generated at S510 for the respective multiple times of light emission and the respective transducers 121. Provided that the plurality of the photoacoustic image data can be generated, the photoacoustic image data may be generated for every light emission, or each piece of the photoacoustic image data may be generated from the projection signals derived from multiple times of light emission.

For example, the computer 150 generates image data that represents the spatial distribution of initial sound pressure p0 for every light emission, based on a projection signal b (ri, t) as defined in Expression (3). Consequently, a plurality of image data for the respective multiple times of light emission are generated, and the plurality of image data can be acquired.

$$\begin{matrix}[ Expression 3 ] & \\ {p}_{0} ( {r}_{0} ) = \frac{{\sum }_{i}^{N} b ( {r}_{i} , t = \frac{\vert {r}_{i} - {r}_{0} \vert}{c} ) \cdot Δ {Ω}_{i}}{{\sum }_{I}^{N} Δ {Ω}_{i}} & Expression ( 3 )\end{matrix}$$

Here, r0 is a position vector that represents a position (also referred to as a reconstruction position or a noteworthy position) of reconstruction, p0 (r0) is the initial sound pressure at the position of the reconstruction, and c is the acoustic velocity on a propagation path. ΔΩI is a solid angle at which the i-th transducer 121 is viewed from the position of the reconstruction, and N is the number of the transducers 121 used for the reconstruction. The Expression (3) means that the projection signal is multiplied by the weight of the solid angle for delay-and-sum (back projection).

According to the present embodiment, the image data can be generated by using the above analytic reconstruction method or model-based reconstruction method.

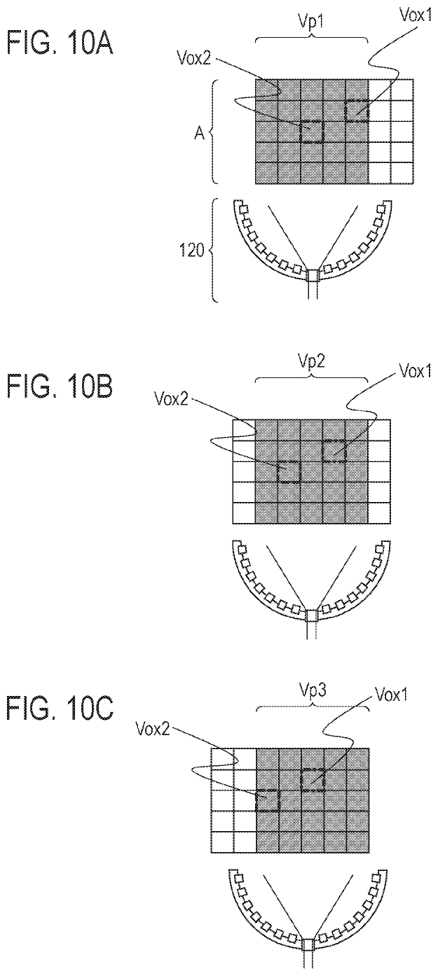

The plurality of the photoacoustic image data that are generated herein correspond to volume data in the case where the range of the reconstruction is a region of a three-dimensional space. The volume data that is generated for every light emission is referred to as partial volume data. For example, as illustrated in FIGS. 10A to 10C, it is considered that the partial volume data is generated while the relative position of the receiver 120 with respect to an imaging region A is changed. In this case, the computer 150 generates partial volume data Vp1 from a signal that is received at light emission at the position illustrated in FIG. 10A. The computer 150 generates partial volume data Vp2 from a signal that is received at light emission at the position illustrated in FIG. 10B. The computer 150 generates partial volume data Vp3 from a signal that is received at light emission at the position illustrated in FIG. 10C. At the same position in the partial volumes illustrated in FIGS. 10A to 10C, a voxel Vox1 is illustrated by using a one-dot chain line, and a voxel Vox2 is illustrated by using a dashed line.

According to the present embodiment, the region of the partial volume data is smaller than the imaging region A. However, the entire imaging region A may be the region in which the partial volume data is generated. That is, the partial volume data having the same region may be generated for every light emission.

(S530: Process of Reading out Template Data)

The computer 150 that serves as template-data-acquiring means reads out template data compatible with the optoacoustic device.

The template data is data on which the spatial sensitivity distribution of the optoacoustic device, which is used for the multiple times of light emission and reception of the photoacoustic waves, is reflected. It can be also said that the template data is data that represents image data that is to be generated when the light absorber is located at a position at each of the multiple times of light emission. That is, it can be also said that the template data corresponds to spatial distribution that represents an estimate image value when the light absorber is located at the position. The template data may be template data on which the properties of the subject 100 are reflected in addition to the spatial sensitivity distribution of the optoacoustic device.

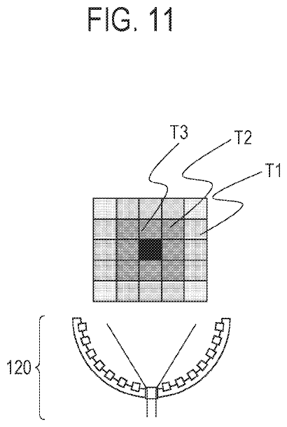

Sensitivity distribution that is exhibited by the transducers 121 that are disposed on the support 122 is read from the RAM or ROM that serves as the storage unit 152. The sensitivity distribution corresponds to data that represents correlation between a relationship in the relative positions of the absorber and the transducers 121 and the image value of partial volume when the absorber at the position is imaged in the case where the absorber is located in a region corresponding to the partial volume. In the case of hemispherical arrangement, the template data exhibits a tendency of the sensitivity distribution to typically become higher as the position is closer to the center of a hemisphere illustrated in FIG. 11 (in the figure, the higher the sensitivity, the darker the color). That is, the template data used according to the present embodiment is spatial distribution data on which the sensitivity of the transducers 121 to the acoustic wave is reflected, based on the relationship in the relative positions of the absorber (voxel that is a target for the reconstruction) and the transducers 121.

The computer 150 may acquire the template data relative to each light emission by assigning a coordinate to predetermined template data, based on information about the relative positions of the receiver 120 and the noteworthy position at the light emission. For example, the computer 150 can generate the template data relative to each light emission by defining the coordinate such that the spatial sensitivity distribution of the optoacoustic device is associated with a position in the receiver 120 at the light emission.

The computer 150 may acquire the information about the relative positions of the receiver 120 and the noteworthy position regarding the multiple times of light emission. Subsequently, the computer 150 may acquire the template data for the multiple times of light emission by reading out the template data relative to the information about the relative positions from the storage unit 152. In this case, the storage unit 152 may store a plurality of the template data relative to the information about the relative positions. In the case where the storage unit 152 does not store the template data relative to the acquired information about the relative positions, new template data may be generated by interpolation with the template data relative to information about relative positions close to the acquired information about the relative positions.

The description herein pertains to a readout method. However, the template data may be calculated by a device in the computer 150 for every imaging. Also, in this case, the effects of the present invention can be achieved. When the template data is calculated, information about the acoustic velocity of the photoacoustic wave on the propagation path or the attenuation characteristics of the acoustic wave may be considered in addition to information about the relative positions of the noteworthy position and the transducers 121. The light intensity distribution of the emission light may be considered for the template data. Diffusion or attenuation due to light propagation in the subject may be considered for the template data. That is, any effect on the image data at each light emission can be considered for the template data. Different template data may be used for every light emission. The same template data may be used for every light emission.

The computer 150 may change the template data depending on the size of the target for observation, for example, the diameter of the blood vessel. That is, the computer 150 may change the template data depending on a reception frequency band or a frequency band used in processing. Specifically, an effect of variation in the directivity of a reception element depending on the frequency of the photoacoustic signal to be received may be reflected on the template data. The computer 150 may change the template data depending on the shape of the target or the optical characteristics of the target. For example, in a case considered herein, the user specifies information (such as the size and shape of the target) about the target. In this case, the computer 150 may acquire the template data relative to the specified information about the target in accordance with information about a relationship between the information about the target and the template data (a relationship table or a relational expression).

(S540: Template Calculation Process)

The computer 150 that serves as correlation-information-acquiring means performs a template calculation process by using the template data and the partial volume data that is generated at multiple positions. Attention is paid to the specific voxel Vox1 to describe the template calculation process. The image value data of a plurality of the partial volume data Vp1, Vp2, and Vp3 at the position of the voxel Vox1 is designated by P1, P2, and P3. Subsequently, data relative to the relative positions thereof is extracted from the template data by using the relationship in the relative positions of the noteworthy position and the transducers 121. Specifically, template data α1 at a position T1 corresponding to the position of the voxel Vox1 of the partial volume data Vp1 in FIG. 10A is extracted. The computer 150 also extracts template data α2 at a position T2 corresponding to the position of the voxel Vox1 of the partial volume data Vp2. The computer 150 also extracts template data α3 at a position T3 corresponding to the position of the voxel Vox1 of the partial volume data Vp3. The computer 150 calculates a correlation value by using the data as following Expression 4.

$$R vox 1 = \frac{{\sum }_{i = 1}^{3} ( Pi \times α i )}{\sqrt{{\sum }_{i = 1}^{3} {Pi}^{2}} \times \sqrt{{\sum }_{i = 1}^{3} α {i}^{2}}} \qquad [ Expression 4 ]$$

Rvox1 is the correlation value relative to the voxel Vox1. i is the light emission number. Pi is the image value of the voxel Vox1 relative to the i-th light emission. αi is the template data of the voxel Vox1 relative to the i-th light emission.

That is, the computer 150 calculates information about correlation relative to the voxel Vox1 by using the image value of the voxel Vox1 and the template data relative to the information about the relative positions of the voxel Vox1 and the transducers 121. At this time, the computer 150 calculates the correlation value relative to the voxel Vox1 by using a plurality of the template data for the multiple times of light emission and the image value data for the multiple times of light emission.

In the case where the number of the volume data from which calculation is made is N, the calculation is made as following Expression 5.

$$R ( X , Y , Z ) = \frac{{\sum }_{i = 1}^{N} ( P ( X , Y , Z , i ) \times α ( xi , yi , zi ) )}{\sqrt{{\sum }_{i = 1}^{N} {P ( X , Y , Z , i )}^{2}} \times \sqrt{{\sum }_{i = 1}^{N} {α ( X , Y , Z , i )}^{2}}} \qquad [ Expression 5 ]$$

R (X, Y, Z) is the result (here, the correlation value) of template calculation relative to the noteworthy position (X, Y, Z) of the volume data. P (X, Y, Z, i) is the image value of the partial volume data at the i-th light emission relative to the noteworthy position (X, Y, Z) of the volume data. α (X, Y, Y) is the value of the template data at the i-th light emission relative to the noteworthy position (X, Y, Z) of the volume data. R (X, Y, Z) ranges from −1 to 1. In the case where the coordinate of the partial volume data does not completely coincide with the coordinate of the template data, the coordinate of either the partial volume data or the template data may be converted into the coordinate of the other. Both of the coordinates of the data may be replaced with a new coordinate. At this time, a value at a coordinate may be replaced with a value at another coordinate, and a value at the other coordinate may be calculated by interpolation. This means that, when attention is paid to a specific position in the volume data, a correlation value between the image value column of partial volume relative to the specific position and a template data column relative to the specific position is calculated. That is, the computer 150 that serves as the correlation-information-acquiring means can acquire information about correlation between the image value column of a plurality of the image data at the noteworthy position and the data column of the plurality of the template data at the noteworthy position.

The template calculation process that is performed over the entire imaging region A enables the result (here, the correlation value) of the template calculation relative to the entire imaging region A to be acquired. The target for the template calculation may be a part of the imaging region A or a region other than the imaging region A.

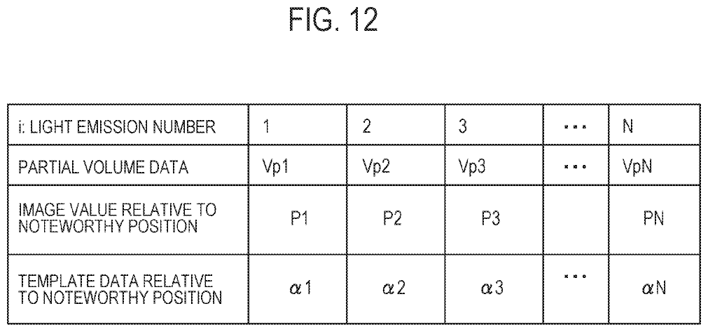

FIG. 12 is a table illustrating relationships among the template data, the image value, and the partial volume data at the i-th light emission (i=1 to N). As illustrated in FIG. 12, the image value and the template data are associated with a common light emission number i. That is, the image value column and the template data column are associated with each other by using the common light emission number. For example, the image value column of the plurality of the image data corresponds to data (for example, matrix data) that contains image values P arranged in order of i ranging from 1 to N. The template data column of the plurality of the template data corresponds to data (for example, matrix data) that contains template data c arranged in order of i ranging from 1 to N. The image value column and the data column may not correspond to the data that contains the image values and the template data arranged in order of i ranging from 1 to N.

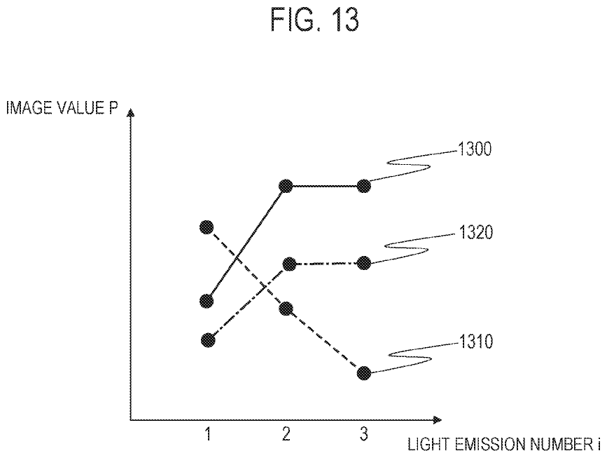

The properties of the correlation value will now be described with reference to FIG. 13. In FIG. 13, the horizontal axis represents the light emission number, and the vertical axis represents the image value at the noteworthy position. In a graph, a solid line 1300 represents changes in the image values of the partial volume data Vp1, Vp2, and Vp3 at the noteworthy position in the case where the light absorber is located at the noteworthy position. In the graph, a dashed line 1310 represents changes in the image values of the template data α1, α2, and α3 at the noteworthy position. In the graph, a one-dot chain line 1320 represents changes in the image values of the partial volume data Vp1, Vp2, and Vp3 at the noteworthy position in the case where the light absorber is not located at the noteworthy position.

In the case where the absorber is located at the noteworthy position, as the noteworthy position with respect to the transducers 121 changes, the image value of the partial volume data relative to the noteworthy position changes together with a change in the template data relative to the noteworthy position. For example, in the case where the absorber is located at the position of Vox1 in FIGS. 10A to 10C, the image value changes as schematically illustrated by the solid line 1300 in FIG. 13.

In the case where the absorber is not located at the noteworthy position (noteworthy voxel), but the absorber is located at a position that differs from the noteworthy position, there is a possibility that an artifact occurs during the image reconstruction of the photoacoustic signal from the absorber that is not located at the noteworthy position and affects the image value at the noteworthy position. For example, in a case considered herein, the position of Vox1 in FIGS. 10A to 10C is the noteworthy position, and the absorber is not located at the position, but the absorber is located at the position of Vox2. In this case, the noteworthy position Vox1 of the partial volume data is affected by an artifact that occurs due to the absorber at Vox2, and the image value changes as schematically illustrated by the dashed line 1310 in FIG. 13.

The template data relative to the relative position of the noteworthy position Vox1 in the partial volume data is illustrated by the one-dot chain line 1320 in FIG. 13.

It can be understood that the correlation value between the data that is represented by the solid line 1300 in the case where the absorber is located at the noteworthy position and the data that is represented by the one-dot chain line 1320 tends to be a positive large value (close to 1). However, it can be understood that the correlation value between the data represented by the dashed line 1310 in the case where the absorber is not located at the noteworthy position and the data that is represented by the one-dot chain line 1320 tends to be a small value, or a negative value. That is, in the case where the absorber is located at the noteworthy position, the correlation value tend to be large. In the case where the absorber is not located at the noteworthy position, the correlation value tends to be small.

The calculation with the template data in this way enables information about the presence or absence of the absorber at each voxel to be acquired. That is, the information about the correlation corresponds to information about the possibility of the presence of the target.

The computer 150 that serves as the display-controlling means may cause the display unit 160 to display an image based on the information about the correlation. The computer 150 may calculate the information about the correlation relative to multiple positions and may cause the display unit 160 to display the spatial distribution of the information about the correlation. The image value may be assigned to the luminance value. The image value may be assigned to a color map for coloring. Combined volume data that is thus acquired is used as the photoacoustic image data.

When the user specifies the desired position by using the input unit 170, the information about the correlation relative to the specified position may be displayed on the display unit 160. In this case, the computer 150 acquires the information about the correlation relative to the position that is determined based on the specification of the user and can cause the display unit 160 to display a text image based on the information about the correlation relative to the position. The computer 150 may read, from the storage unit 152, the information about the correlation relative to the specified position that is calculated in advance. After the position is specified, the computer 150 may calculate the information about the correlation relative to the specification. The user may specify a position in the image of the partial volume data that is displayed on the display unit 160, in an image of combination volume data described later, in an image that is generated by another modality, or in another image.

Since the information about the correlation thus corresponds to the information about the possibility of the presence of the target, displaying the image based on the information about the correlation makes it easy for the user to determine whether the target is located at the noteworthy position.

(S550: Combination Process of Partial Volume)

The computer 150 that serves as the image-data-generating means generates the combination volume data (combination image data) by performing a combination process of combining a plurality of the partial volume data. The computer 150 extracts the image value at the same position in the imaging region A, that is, at the same voxel (for example, Vox1) from the plurality of the partial volume data. The computer 150 combines the partial volume data by adding or averaging the extracted image value and calculates the combination volume data relative to the imaging region A. The computer 150 generates the combination volume data of the imaging region A by performing the combination process for each voxel relative to the imaging region A. The combination process may not necessarily be performed for the voxels relative to the entire imaging region A. The combination process may be performed for the voxels relative to multiple positions in a region that the user desires.

(S560: Generation of Combined Volume Data)

The computer 150 that serves as the image-data-generating means generates the combined volume data by using the combination volume data and the result of the template calculation. The combined volume data is weighted combination volume data that is acquired by weighting the combination volume data by using the information about the correlation that is the result of the template calculation.

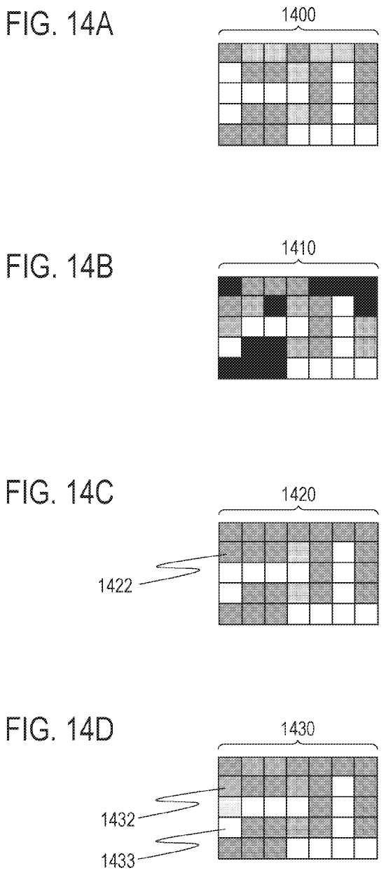

The content of weighting calculation in this process will now be described with reference to FIGS. 14A to 14D.

FIG. 14A schematically illustrates combination volume data 1400 of a region. In the combination volume data 1400, as the degree of brightness increases (color is closer to white), the image value increases.

FIG. 14B schematically illustrates a template calculation result (spatial distribution of the information about the correlation) 1410 at the same position as with the combination volume data 1400. In the template calculation result 1410, as the degree of brightness increases (color is closer to white), the image value increases.

The computer 150 can cause the display means to selectively display the combination volume data relative to a position at which the value of the template calculation result 1410 satisfies a predetermined condition. FIG. 14C illustrates an example of combined volume data (weighted combination volume data) 1420. The combined volume data 1420 illustrated in FIG. 14C corresponds to image data that is generated by selectively using the image value of the combination volume data at a position at which the value of the template calculation result 1410 is equal to or more than a threshold. In FIG. 14C, the predetermined condition means being equal to or more than the threshold. The combined volume data 1420 corresponds to image data in which a small image value, for example, 0 is inputted for a voxel at which the value of the template calculation result 1410 is equal to or less than a predetermined value. That is, the combined volume data 1420 represents the result of weighting the combination volume data 1400 by using the template calculation result 1410. As a result of weighting in this way, for example, at a voxel 1422, the image value of a combined volume is large, but the template calculation result is small. Accordingly, in the combined volume data, the image value at the position of the voxel is the small value. The threshold for the template calculation result is thus defined and used as a mask. Consequently, the image value makes it easy to visually recognize a region in which the absorber is highly possibly located. The threshold may be determined to be a value that is read by the computer 150 from a memory. The computer 150 may determine the threshold is a value that is specified by the user. The computer 150 may cause the display unit 160 to display a recommended value of the threshold to assist the user in specifying the threshold. In this case, the computer 150 may determine the recommended value of the threshold based on information about the target for observation and may cause the display unit 160 to display the recommended value.

FIG. 14D illustrates another example of combined volume data (weighted combination volume data) 1430. The image value of the combination volume data is weighted such that the weight is large for a voxel (for example, a voxel 1433) at which the correlation of the template calculation result is strong, and the weight is small for a voxel (for example, a voxel 1432) at which the correlation of the template calculation result is weak. This process may be multiplication or may be weighting in accordance with a predetermined relationship table or relational expression (table or expression that defines a relationship between the correlation and the weight). In this case, the relationship table or the relational expression may be such that the stronger the correlation, the larger the weight. In the combination volume data, the voxel 1433 and the voxel 1432 have the same image value. However, after such a process, the voxel 1432 at which the correlation is weak has a small image value in the combined volume data 1430, and the voxel 1433 at which the correlation is strong has a large image value in the combined volume data 1430. Consequently, the image value makes it easy to visually recognize the voxel 1432 at which an artifact highly possibly occurs and the voxel 1433 at which the absorber is highly possibly located.

In an example described above, the threshold for the template calculation result is set for weighting. In another example described above, the template calculation result is used as the weight for weighting. However, these two kinds of processes may be combined. For example, when the template calculation result is a predetermined value or less, the combined volume data is set to 0, and when the combined volume has the template calculation result that is more than the predetermined value, a weight based on the template calculation result may be used for the combination volume data to determine the image value.

The image value may be assigned to the luminance value. The image value may be assigned to the color map for coloring. The combined volume data that is thus acquired is used as the photoacoustic image data. At S600, an image based on the combined volume data corresponding to the photoacoustic image data is displayed on the display unit 160.

In accordance with the instruction of the user, the computer 150 may switch between displaying the image based on the combined volume data (weighted combination volume data) and displaying an image based on the combination volume data that is not weighted. The computer 150 may alternately switch between these images automatically. In this case, the computer 150 may cause the volume data on which the displayed image is based to be displayed on a screen.

The computer 150 may change a weight between the combined volume data (weighted combination volume data) and the combination volume data that is not weighted for display. In this case, the computer 150 may cause the degree of the weight for combination to be displayed on the screen.

In FIG. 14C, the voxel at which the value of the template calculation result 1410 is equal to or less than the predetermined value has a small image value. The predetermined value may be changed depending on the instruction of the user or a subject to be imaged. In this case, the predetermined value may be displayed on the screen.

The computer 150 may change the transparency of the combination volume data depending on the correlation value to generate the combined volume data for display. For example, the combined volume data may be generated such that the transparency of the combination volume data increases as the correlation value decreases.

The computer 150 may superimpose an image a color map of which is made by using the correlation value on the combined volume data or on the combination volume data that is not weighted. The computer 150 may assign brightness to the combined volume data or to the combination volume data that is not weighted and may generate a color image in which a color (hue or saturation) is assigned to the correlation value for display.

The computer 150 can generate an image in which a real image and an artifact are identifiable by using the image value column of the plurality of the image data at the noteworthy position and the template data column of the plurality of the template data at the noteworthy position as described above.

The computer 150 may change a method of displaying these depending on information (for examples, the subject to be imaged or a clinical department) associated with the photoacoustic image data, or the instruction of the user.

According to the present embodiment, the correlation value between the template data and the change in the image value at the noteworthy position is used as the result (the information about the correlation) of the template calculation. However, the result (the information about the correlation) of the template calculation may be an inner product value that can be calculated by the following Expression 6.

R′(X,Y,Z)=Σᵢ₌^{N}(P(X,Y,Z,i)×α(xi,yi,zi)) [Expression 6]

It may be determined that there is correlation if a positive peak of a cross-correlation coefficient that can be calculated by the Expression 7 below appears when there is no data column deviation (that is, j=0), and this may be used as the result of the template calculation. For example, if the positive peak appears when there is no data column deviation (j=0), 1 may be substituted for the result of the template calculation, and if not, 0 or a value of less than 1 may be substituted.

$$R ( X , Y , Z , j ) = \frac{{\sum }_{i = 1}^{N} ( P ( X , Y , Z , i + j ) \times α ( xi , yi , zi ) )}{\sqrt{{\sum }_{i = 1}^{N} {P ( X , Y , Z , i + j )}^{2}} \times \sqrt{{\sum }_{i = 1}^{N} {α ( xi , yi , zi )}^{2}}} \qquad [ Expression 7 ]$$

These values are calculated to represent the degree to which the template data and the image value at the noteworthy position change in the same way. Provided that the correlation, or relationship between the template data and the image value at the noteworthy position can be evaluated, the use of another indicator also enables the effects of the present invention to be achieved.

According to the present embodiment, the expression outputs the correlation value by using the template data and the partial volume data relative to the noteworthy position. Whenever the partial volume data is acquired, each voxel of the partial volume data may be multiplied by the spatial distribution (that is, the template data) that represents the estimate image value when the light absorber is located at the position of the voxel, and the product thereof may be combined. Also, in this case, the same calculation result as in the present embodiment can be acquired. Specifically, whenever the partial volume data is acquired (that is, whenever i increases), calculation is made as following Expression 8.

RN(X,Y,Z,i)=P(X,Y,Z,i)×α(xi,yi,zi)

RD1(X,Y,Z,i)=P(X,Y,Z,i)²

RD2(X,Y,Z,i)=α(xi,yi,zi)² [Expression 8]

In addition, data is updated for every voxel by the following Expression 9.

RNs(X,Y,Z)=Σᵢ₌₁^{K}RN(X,Y,Z,i)

RD1s(X,Y,Z)=Σᵢ₌₁^{K}RD1(X,Y,Z,i)

RD2s(X,Y,Z)=Σᵢ₌₁^{K}RD2(X,Y,Z,i) [Expression 9]

When the required number of the partial volume data is acquired, the result of the template calculation is acquired by the following Expression 10.

$$R ( X , Y , Z ) = \frac{RNs ( X , Y , Z )}{\sqrt{RD 1 s ( X , Y , Z )} \times \sqrt{RD 2 s ( X , Y , Z )}} \qquad [ Expression 10 ]$$

The use of such a method enables a data amount that is needed during calculating to be decreased. Consequently, an increased amount of data can be calculated with a decreased size of a system.

In an example described according to the present embodiment, the computer 150 generates the image data to acquire the image data. However, the computer 150 may acquire the image data by reading out the image data that is stored in a storage device in advance. The storage device that stores the image data may be a storage server such as a PACS (Picture Archiving and Communication System) via a network. The storage device that stores the image data may be a storage unit of the computer 150.

In the case where the spatial sensitivity distribution changes during the multiple times of measurement, the present invention can be used. For example, in the case where the position of the light emission changes or the position at which the photoacoustic wave is received changes during the multiple times of light emission, and the spatial sensitivity distribution consequently changes, the present invention can be used for the optoacoustic device.

In an example described according to the above embodiment, the present invention is used for the optoacoustic device. However, the modality for which the present invention can be used is not limited to the optoacoustic device. That is, the present invention can be used for image data that is generated by any modality in which an artifact occurs. Examples of the modality for which the present invention can be used include an ultrasonic diagnosis apparatus, an MRI apparatus, and an X-ray CT apparatus. Also, in the case of application to such a modality, the information-processing apparatus can acquire the information about correlation between the image column of a plurality of image data relative to multiple timings of measurement and the template data column of a plurality of template data relative to multiple times of measurement. The measurement described herein means measurement (imaging) that is needed to generate one frame of image data. For example, in the case of the ultrasonic diagnosis apparatus, measurement that is carried out once corresponds to transmission and reception of an ultrasonic wave to generate one frame of image data. Such a method of processing information makes it easy to determine whether the possibility of the presence of the target (target for observation) at the noteworthy position in an image is high or low.

An information-processing apparatus according to the present invention can acquire information that makes it easy to determine whether the possibility of the presence of a target (target for observation) at a noteworthy position in an image is high or low.

### Other Embodiments

The embodiments have been described above in detail. Note that the present invention can be implemented as a system, an apparatus, a method, a program, a storage medium, or the like. More specifically, for example, the present invention may be applied to a system including a plurality of devices among which the functions of the image processing apparatus are distributed. Conversely, the present invention may be applied to an apparatus including a single device. A function and/or a process according to the present invention may be realized on a computer by installing a program code on the computer thereby realizing the present invention. The computer program itself for realizing the functions and/or the processes disclosed in the above-described embodiments also falls within the scope of the present invention. The functions of the above-described embodiments are implemented by a computer by reading a program and executing it. Furthermore, a function of an embodiment may also be realized by performing an operation in cooperation with an OS or the like running on the computer based on an instruction of the program. In this case, the OS or the like performs part or all of the actual processing thereby realizing the functions of the above-described embodiments. Furthermore, the program read from a storage medium may be written into a memory provided in a function expansion board inserted in the computer or a function expansion unit connected to the computer, and some or all of the functions of the above-described embodiments may be realized. Note that the scope of the present invention is not limited by the above-described embodiments.

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

The present invention is not limited to the above embodiments. Various modifications and alterations can be made without departing from the spirit and scope of the present invention. Accordingly, the following claims are attached to make the scope of the present invention public.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

What is claimed is:

1. An information-processing apparatus that processes information based on a photoacoustic wave that is generated from a subject by light emission to the subject, the information-processing apparatus comprising:
an image-data-acquiring means that acquires a plurality of image data for multiple times of light emission, based on a photoacoustic wave that is generated by the multiple times of light emission to the subject;
template-data-acquiring means that acquires a plurality of template data for the multiple times of light emission; and
correlation-information-acquiring means that acquires information about correlation between an image value column of the plurality of image data at a noteworthy position and a template data column of the plurality of template data at the noteworthy position.

2. The information-processing apparatus according to claim 1, wherein each of the plurality of template data is template data on which the multiple times of light emission and spatial sensitivity distribution of an optoacoustic device that receives the photoacoustic wave are reflected.

3. The information-processing apparatus according to claim 1, wherein each of the plurality of template data corresponds to an estimate image value of image data that is to be generated when a light absorber is located at the noteworthy position at each of the multiple times of light emission.

4. The information-processing apparatus according to claim 1, wherein the template-data-acquiring means acquires the plurality of template data by assigning a coordinate to predetermined template data, based on information about a relative position of reception means that receives a photoacoustic wave and the noteworthy position regarding the multiple times of light emission.

5. The information-processing apparatus according to claim 1, wherein the template-data-acquiring means acquires the plurality of template data in a manner in which information about a relative position of reception means that receives a photoacoustic wave and the noteworthy position is acquired regarding the multiple times of light emission, and template data relative to the information about the relative position is read out from storage means.

6. The information-processing apparatus according to claim 1, wherein an image value that is included in the image value column and template data that is included in the template data column are associated with common light emission.

7. The information-processing apparatus according to claim 1, wherein the correlation-information-acquiring means calculates the information about the correlation by an expression defined as:
$$R ( X , Y , Z ) = \frac{{\sum }_{i = 1}^{N} ( P ( X , Y , Z , i ) \times α ( xi , yi , zi ) )}{\sqrt{{\sum }_{i = 1}^{N} {P ( X , Y , Z , i )}^{2}} \times \sqrt{{\sum }_{i = 1}^{N} {α ( X , Y , Z , i )}^{2}}} \qquad [ Expression 1 ]$$
where (X, Y, Z) is a coordinate of the noteworthy position, i (i=1 to N) is a light emission number, R is the information about the correlation, P is the image value, and a is the template data.

8. The information-processing apparatus according to claim 1, further comprising: display-controlling means that causes display means to display an image based on the information about the correlation.

9. The information-processing apparatus according to claim 1, wherein the image-data-acquiring means generates combination image data by combining the plurality of image data and generates weighted combination image data by weighting the combination image data by using the information about the correlation.

10. The information-processing apparatus according to claim 9, further comprising: display-controlling means that causes display means to display an image based on the weighted combination image data.

11. The information-processing apparatus according to claim 1, further comprising: display-controlling means that causes display means to selectively display image data relative to a position at which the correlation satisfies a predetermined condition.

12. The information-processing apparatus according to claim 1, wherein the display-controlling means sets the predetermined condition that a user specifies by using input means.

13. The information-processing apparatus according to claim 1, wherein the plurality of image data are generated such that a position of the light emission or a position at which a photoacoustic wave is received changes at each of the multiple times of light emission.

14. An information-processing apparatus that processes image data that is generated by a modality, the information-processing apparatus comprising:
image-data-acquiring means that acquires a plurality of image data for multiple times of measurement;
template-data-acquiring means that acquires a plurality of template data for the multiple times of measurement; and
correlation-information-acquiring means that acquires information about correlation between an image value column of the plurality of image data at a noteworthy position and a template data column of the plurality of template data at the noteworthy position.

15. A method of processing information based on a photoacoustic wave that is generated from a subject by light emission to the subject, the method comprising:
acquiring a plurality of image data for multiple times of light emission, based on a photoacoustic wave that is generated by the multiple times of light emission to the subject;
acquiring a plurality of template data for the multiple times of light emission; and
acquiring information about correlation between an image value column of the plurality of image data at a noteworthy position and a template data column of the plurality of template data at the noteworthy position.

16. A non-transitory computer-readable medium storing program for causing a computer to execute the method of processing information according to claim 15.
